(19) United States
(12) Patent Application Publication
(10) Pub. No.: US 2020/0234823 A1
(43) Pub. Date: Jul. 23, 2020

(54) HEALTH MONITORING USING SOCIAL RHYTHMS STABILITY

(71) Applicant: Cornell University, Ithaca, NY (US)

(72) Inventors: Saeed Abdullah, Ithaca, NY (US); Mark Matthews, Ithaca, NY (US); Tanzeem Choudhury, Pittsford, NY (US)

(21) Appl. No.: 16/821,908

(22) Filed: Mar. 17, 2020

Related U.S. Application Data
(63) Continuation of application No. 15/458,869, filed on Mar. 14, 2017, now abandoned.
(60) Provisional application No. 62/308,117, filed on Mar. 14, 2016.

(51) Int. Cl. G16H 40/67; G16H 40/63

(52) U.S. Cl. CPC G16H 40/67; G16H 40/63

(57) ABSTRACT

Methods, systems, and devices are disclosed for passively monitoring a health condition of a subject using a mobile device. In some aspects, a method includes producing a set of quantitative metrics based on one or more parameters including location, movement, and sound obtained from the mobile device, where the produced set of quantitative metrics includes a location cluster value, a travel distance value, a frequency of conversation value, and an activity value, each of the quantitative metrics over a respective predetermined time period; and processing the set of quantitative metrics to determine a metric indicative of a current clinical state of the subject in connection with one or more measures including daily routines, mood or energy of the subject. Using the sensed parameters, rhythmicity markers and/or departure from stability can be predicted to alert a caregiver of the patient's health status, such as a current mental health state.

### CROSS REFERENCE TO RELATED APPLICATIONS

This patent document is a continuation of U.S. patent application Ser. No. 15/458,869, entitled “HEALTH MONITORING USING SOCIAL RHYTHMS STABILITY” filed on Mar. 14, 2017 which claims priorities to and benefits of U.S. Provisional Patent Application No. 62/308,117 entitled “MENTAL HEALTH MONITORING USING SOCIAL RHYTHMS STABILITY” filed on Mar. 14, 2016. The entire content of the aforementioned patent application is incorporated by reference as part of the disclosure of this patent document.

### STATEMENT REGARDING FEDERALLY SPONSORED RESEARCH OR DEVELOPMENT

This invention was made with government support under grant NIH 5R01MH103148-03 awarded by the National Institutes of Health. The government has certain rights in the invention.

### TECHNICAL FIELD

This patent document relates to systems, devices, and processes that use ambient light and sound sensing for determining a user's social rhythms stability and mental health state.

### BACKGROUND

Mental illness is characterized as a medical condition that affects a person's thinking, feeling, behavior, or mood. A mental illness condition can affect a person's ability to socially relate to others and generally function each day in life. A mental illness condition is typically diagnosed by a mental health professional based on a behavioral or mental pattern adversely affects the person's ability to function in their everyday life. Features of the person's behavioral or mental patterns associated with a mental illness may be persistent, relapsing and remitting, or occur as a single episode. Onset of episodes of certain mental health conditions are hard to detect, and each person with a mental illness condition will experience that condition differently, even among people with the same diagnosis.

### SUMMARY

In some aspects, a method for passively monitoring a health condition of a subject using a mobile device includes producing a set of quantitative metrics based on one or more parameters including location, movement, and sound obtained from a mobile communications device associated with the subject, in which the produced set of quantitative metrics includes a location cluster value, a travel distance value, a frequency of conversation value, and an activity value, and each of the quantitative metrics is over a respective predetermined time period; and processing the set of quantitative metrics to determine a metric indicative of a current clinical state of the subject in connection with one or more measures including daily routines, mood or energy of the subject.

In some aspects, a device for passively monitoring a health condition includes a plurality of sensors including a location sensor, a motion sensor and a sound sensor, in which the sensors detect location data, movement data, and sound data associated with a user of the device; and a data processing unit including a memory to store data from the sensors and a processor configured to processes the location data, the movement data and the sound data to generate a set of quantitative metrics including a location cluster value, a travel distance value, a frequency of conversation value, and an activity value, in which each of the quantitative metrics is over a respective predetermined time period, and to determine a metric indicative of a current clinical state of the user in connection with one or more measures including daily routines, mood or energy of the user, in which the metric indicative of the current clinical state is determined at least partly without active input from the user.

In some aspects, a user device that includes a memory, a processor, an ambient light sensor, and a microphone to receive ambient audio is disclosed. The ambient light sensor senses ambient light and produces an ambient light signal. The microphone periodically samples ambient audio to produce sampled audio, and the processor uses the ambient light signal and the sampled audio to determine a present mental health condition of a user of the user device.

In some aspects, a method of monitoring a patient's current condition, implemented by a user device is disclosed. The method includes sensing a physical parameter related to the patient's ambience. The method includes tracking usage data of the patient's use of the user device. The method includes estimating, at least partly without explicit input from the patient, the patient's current condition from the physical parameter and the usage data.

The subject matter described in this patent document can be implemented in specific ways that provide one or more of the features described herein.

### BRIEF DESCRIPTION OF THE DRAWINGS

FIG. 1A shows a diagram of an example system architecture in accordance with embodiments of the present technology.

FIG. 1B shows a diagram of an example method for passively monitoring mental health of a subject using a mobile communications device such as a smartphone.

FIG. 1C illustrates a flowchart for an example method of mental health monitoring.

FIG. 2 shows a diagram of an example block diagram of a data processing unit in accordance with embodiments of the present technology.

FIG. 3 shows a diagram of an example software architecture for processing passively monitored data and characterizing social rhythms of a user in accordance with embodiments of the present technology.

FIG. 4 shows a diagram of an example data collection module of the software application in accordance with embodiments of the present technology.

FIG. 5 shows a diagram of an example embodiment of a motion data processing module.

FIG. 6 shows a diagram of an example embodiment of a location data processing module.

FIG. 7 shows a diagram of an example embodiment of a device usage data processing module.

FIG. 8 shows a diagram of an example embodiment of a sound data processing module.

FIG. 9 shows a diagram of an example embodiment of a sleep analysis module.

FIG. 10 shows an example of a paper-based Social Rhythm Metric (SRM) form that is used as part of Interpersonal Social Rhythm Therapy (ISRT) process.

FIG. 11 shows screenshots of an example user interface of the customized smartphone app used in the example study.

### DETAILED DESCRIPTION

Bipolar disorder is a serious mental illness that has been recognized as one of the eight leading causes of years lived with disability worldwide. Bipolar disorder affects approximately 2.6% of the US population aged 18 and older in a given year. It is associated with poor functional and clinical outcomes and high suicide rates. It also induces huge societal cost the direct and indirect cost associated with bipolar disorder I and II in 2009 has been estimated to be $151 billion in United States alone.

Bipolar disorder is characterized by disturbances in rhythmicity. A number of theories link disturbances in social rhythms, such as changes in sleep timing and other routines to mood episodes for individuals with bipolar disorder. The Social Zeitgeber hypothesis suggests that certain life events may lead to episode onset due to their effect on individuals' social routines. Changes in routine, in turn, affect endogenous circadian rhythms leading to mood symptoms and, in vulnerable individuals, to mood episodes.

While there is no cure for bipolar disorder, effective management can reduce the symptoms and result in better prognosis over time. Substantial evidence indicates that interventions targeting social rhythms, sleep-wake rhythms, and light-dark exposure may markedly improve outcomes. Interpersonal Social Rhythm Therapy (IPSRT) is a psychosocial therapy specifically devised to help individuals with bipolar disorder maintain stable daily social rhythms. Increased regularity of social routines is associated with symptomatic improvement and significantly longer intervals between episodes. The work of IPSRT also includes improving interpersonal relations but focuses on the timing of social events for establishing regular social rhythms. To establish and keep track of daily routines, mood and energy, patients use the Social Rhythm Metric (SRM). The SRM, originally developed as a 17-item scale to quantify rhythms of daily life, has subsequently been tested and used as a 5-item therapeutic self-monitoring tool in evidence-based psychosocial interventions. The reduction of the 17-item scale SRM to a 5-item scale SRM was motivated, in part, to accommodate the cumbersome responsibilities and requirements of a user manually tracking and recording their routines, mood, energy levels, etc. associated with SRM.

While the SRM (e.g., including the reduced 5-item scale SRM) has proven effective for assessing stability and rhythmicity of social routines, its paper-and-pencil format has multiple disadvantages as a clinical tool. Longitudinal self-tracking is difficult particularly in light of the inherent characteristics of bipolar disorder. Even well-intentioned patients often forget to complete it. Additionally, in certain stages of illness, momentary and retrospective recall can be particularly challenging for patients with severe psychiatric disorders, and are sometimes unreliable. Nor is the paper format conducive to summarizing collected data, for example creating a visual representation of trends over time that could be used in treatment to enhance patients' self-awareness of their social rhythms. Such issues with paper-and-pencil based tools are well known. For example, it was found that for symptom journaling, patients with bipolar disorder preferred a handheld PDA to paper, reported feeling less social stigma and enjoyed having a more involved role in their treatment. Furthermore, no tools currently exist for accurate sensing of a patient's ambient conditions such as the sight and sound to which the patient is exposed.

Disclosed are systems, methods and devices to passively monitor and assess health conditions using social rhythm stability metrics, including mental health conditions and other clinical conditions such as stroke, Parkinson's disease, and myoclonic epilepsy, among others. The systems, methods and devices in accordance with the present technology are capable of determining social rhythm metric through passive sensing of the user's environment and behavior, thereby not requiring active participation of the user. As such, the present technology described herein can be used to overcome the above-discussed technical problems associated with monitoring and managing mental illness conditions, such as bipolar disorder.

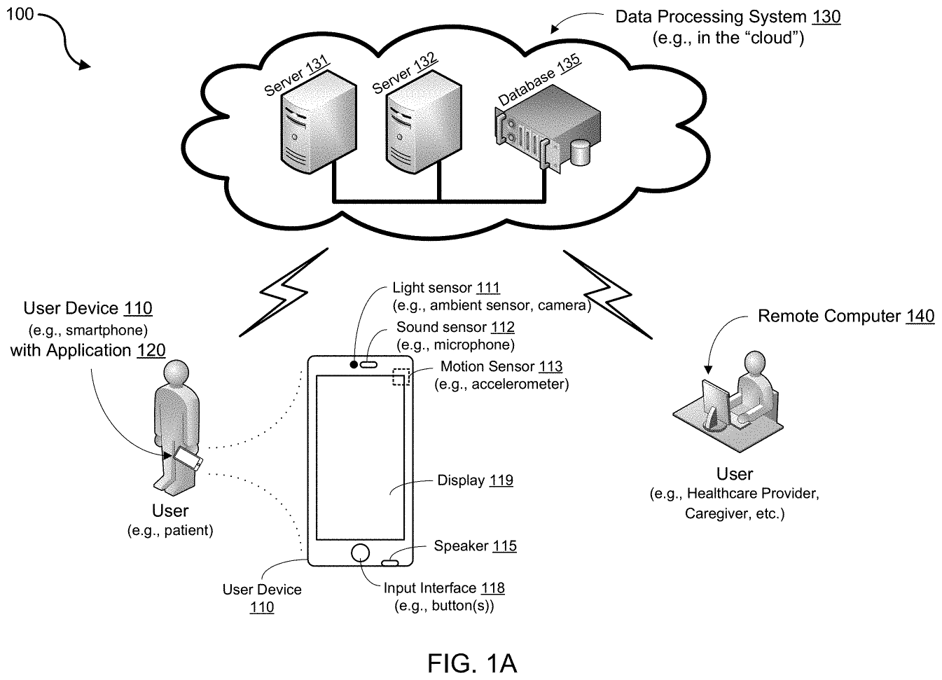

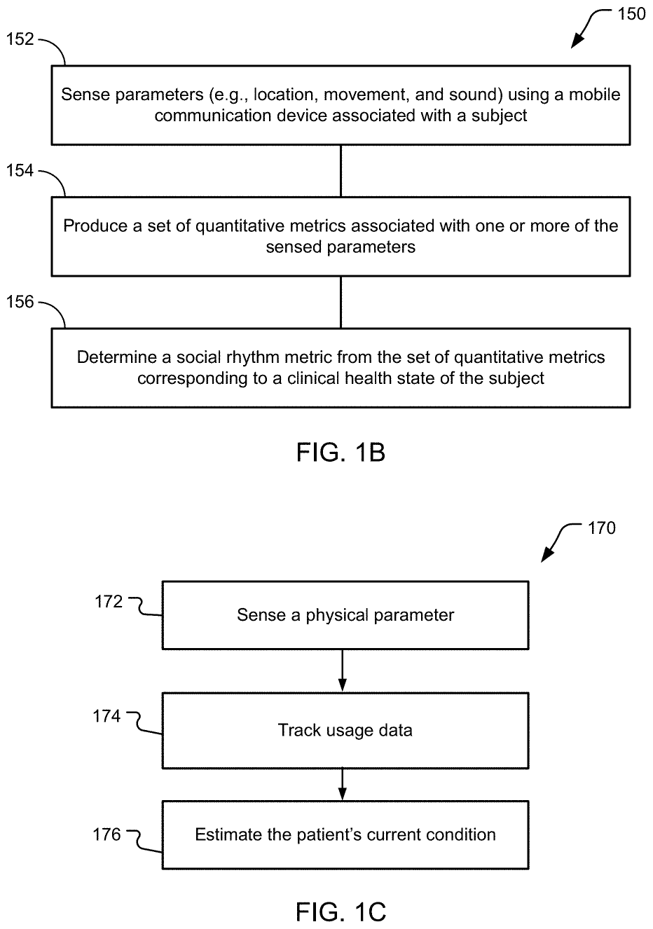

FIG. 1A shows a diagram of an example embodiment of a system 100 for passively monitoring and inferring a current clinical state or condition based on SRM determined in real-time in accordance with the present technology. The system 100 includes a customized software application (“app”) 120 resident on a user device 110, e.g., a mobile computing device such as a smartphone, tablet, smartwatch, etc., or another computing device that is mobile with the user. The app 120 includes program code that is stored in the memory of the user device and executable by a processor of the user device to control the operations of the user device, including various modules and components, for passively monitoring and assessing a mental health condition of the user of the user device. The app 120 is configured to aggregate data from the user device 110, such as device usage data, location data, user input data (e.g., directly and indirectly input to the app 120), and data detected by sensors resident on the user device, e.g., such as a camera, microphone, accelerometer or rate senor, and the like. The app 120 is configured to control at least some functionalities of at least some functional units of the user device 110, such as the aforementioned sensors.

Various embodiments of the user device 110, such as a smartphone, tablet and/or smartwatch or smartglasses, include one or more sensor devices. As shown in the example of FIG. 1A, in which the user device 110 is embodied as a smartphone, the system 110 can employ the use of a light sensor 111 to passively acquire light or image data, a sound sensor 112 to passively acquire audio data, and/or a motion sensor 113 to passively acquire movement data of the user device 110 that pertains to its user (the patient user) that is informative to the SRM in accordance with the present technology. In some embodiments, the light sensor 111 senses ambient light and produces an ambient light signal, and the sound sensor 112 periodically samples ambient sound to produce ambient sound signal, in which the processor of the user device 110 uses the ambient light signal and the ambient sound signal to determine a present mental health condition of the user of the user device 110.

For example, the system 100, via the app 120, can obtain light data indicative of an amount of ambient light present upon the user device 110 from the camera or other light sensor of the user device 110. Similarly, the app 120 can obtain other forms of light-based data from images captured by the camera. In some implementations, the system 100 can obtain and process image data captured by the camera for monitoring or identifying behavior of the patient user, e.g., activity, whereabouts, or interactions by the user. The system 100, via the app 120, can obtain audio data indicative of an amount of ambient sound present upon the user device 110 from the microphone or other sound sensor of the user device 110. The system 100, via the app 120, can obtain motion data indicative of movements of the user device 110 (associated with its user) from one or more motion sensors of the user device 110, e.g., accelerometers, rate sensors, etc.

In some implementations, the system 100, via the app 120, can obtain user input data indirectly from other applications or the operating system of the user device 110, and/or directly based on user interaction with a user interface of the app 120 presented using other functional components of the user device 110 such as the display 119, speaker 115, and/or user input interface(s) 118, such as buttons on the device. In some implementations, the system 100, via the app 120, can obtain device usage data about time, frequency, duration, and/or type of use the user device 110 undergoes. For example, the device usage data can be obtained from data files (e.g., usage logs) associated with the functional units of the user device 110 such as the speaker 115, the display 119, the motion sensor 113, a GPS unit, the camera, the microphone or other unit of the device. Additionally or alternatively, for example, the device usage data can be obtained from other software applications operated by the patient on the user device 110.

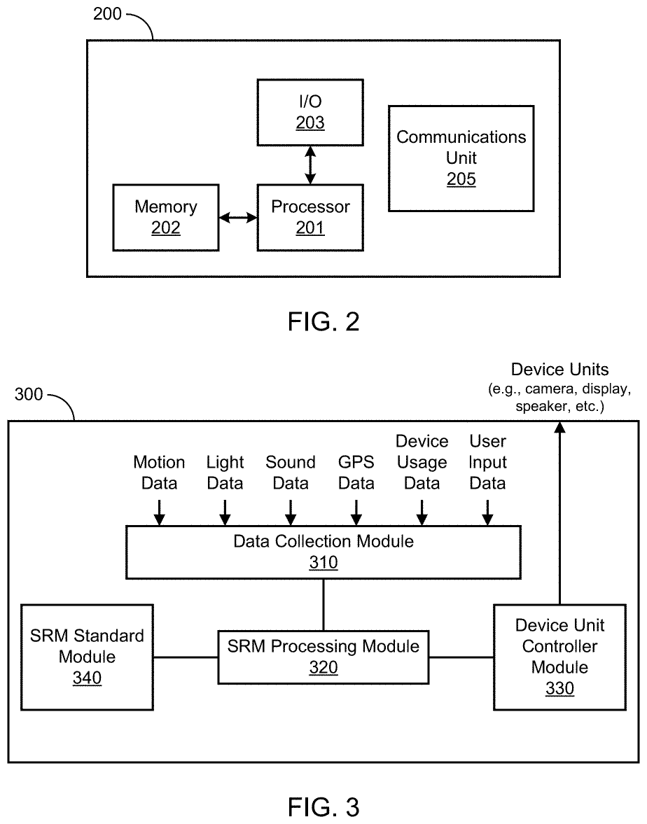

The user device 100 includes a data processing unit 200, shown in FIG. 2, to implement the operations of the app 120 associated with embodiments of the systems and methods for passively monitoring and assessing and/or predicting a mental state of a user based on determining social rhythm metrics in real-time. FIG. 2 shows a diagram of an example block diagram of the data processing unit 200. The data processing unit 200 can include a processor 201 to process data, and a memory 202 in communication with the processor 201 to store and/or buffer data. For example, the processor 201 can include a central processing unit (CPU) or a microcontroller unit (MCU). For example, the memory 202 can include and store processor-executable code, which when executed by the processor 201, configures the data processing unit 200 to perform various operations, e.g., such as receiving information, commands, and/or data, processing information and data, and transmitting or providing information/data to another device. To support various functions of the data processing unit 200, the memory 202 can store information and data, such as instructions, software, values, images, and other data processed or referenced by the processor 201. For example, various types of Random Access Memory (RAM) devices, Read Only Memory (ROM) devices, Flash Memory devices, and other suitable storage media can be used to implement storage functions of the memory 202. In some implementations, the data processing unit 200 includes an input/output unit (I/O) 203 to interface the processor 201 and/or memory 202 to other modules, units or devices associated with the data processing system 130, and/or external devices. The data processing unit 200 includes a communications unit 205, e.g., such as a transmitter (Tx) or a transmitter/receiver (Tx/Rx) unit. The I/O 203 can interface the processor 201 and memory 202 with the communications unit 205 to utilize various types of wired or wireless interfaces compatible with typical data communication standards, for example, which can be used in communications of the data processing unit 200 with other devices, e.g., such as between the app 120 on the user device 110 and the one or more computers of the data processing system 130. Example wireless data communication standards include, but are not limited to, Bluetooth, Bluetooth low energy (BLE), Zigbee, IEEE 802.11, Wireless Local Area Network (WLAN), Wireless Personal Area Network (WPAN), Wireless Wide Area Network (WWAN), WiMAX, IEEE 802.16 (Worldwide Interoperability for Microwave Access (WiMAX)), 3G/4G/5G/LTE cellular communication methods, and parallel interfaces. In some implementations, the data processing unit 200 can interface with other devices using a wired connection via the I/O of the data processing unit 200. The data processing unit 200 can also interface with other external interfaces, sources of data storage, and/or visual or audio display devices, etc. to retrieve and transfer data and information that can be processed by the processor 201, stored in the memory 202, or exhibited on an output unit of the user device 110 (e.g., speaker 115 and/or display 119 of the smartphone) or an external device. In some implementations, for example, the data processing unit 200 is embodied on the one or more computing devices (e.g., in the cloud) of the data processing system 130 in addition or alternatively to the app 120 resident on the user device 110.

Referring back to FIG. 1A, the system 100, in some embodiments, for example, includes a data processing system 130 including one or more computing devices in a computer system or communication network accessible via the Internet (referred to as “the cloud”), e.g., including servers and/or databases in the cloud. In some implementations, the computing devices of the data processing system 130 include one or more servers 131, 132 in communication with each other and one or more databases 135. The data processing system 130 is in communication with the app 120 resident on the user device 110 to receive data and manage processing and storage of the data, such as the data obtained by the sensor modules of the user device 110 and/or SRM data processed by the app 120 on the user device 110. In some implementations, for example, the app 120 can transfer the data to the one or more computing devices of the data processing system 130 using various communication protocols, e.g., including a wired or a wireless communication protocol such as LTE, Wi-Fi, or other.

In some embodiments, for example, the system 100 can include a remote computer 140 to remotely monitor the processed SRM data associated with the user (e.g., patient) obtained by the sensor modules of the user device 110, processed by the app 120, and transferred to the data processing system 130. For example, the remote computer 140 can include a personal computer such as a desktop or laptop computer, a mobile computing device such as a smartphone, tablet, smartwatch, etc., or another computing device. For example, a user of the remote computer 140 can include a healthcare provide such as a doctor, nurse or other, a caregiver of the patient user such as a family member or friend, or healthcare system operator such as user associated with a healthcare payer or managed care facility. In some implementations, the remote computer 140 is provided reports, alerts, or other information associated with the processed SRM data of the patient user.

The system 100 provides multiple advantages over conventional devices and systems to assess clinical or health states of patient subjects. The system 100 provides increases in efficiency of computing and data communication resources on computer systems of its users. For example, the system 100 provides a centralized computer system to collect and analyze data and perform actions based on the analyzed data that reduces excess computational resources on client computing devices (e.g., user device 110 and server computers 131, 132), as well as reduces network traffic that in turn increases efficiencies on the communication infrastructure of the Internet e.g., reducing data traffic between patient users and clinician users. Such increased efficiencies in computing technologies are created while also providing significant improvements and tools for patient users to individually manage complexities associated with continuously assessing and treating a clinical condition, e.g., such as a mental health disorder like bipolar disorder.

The system 100 automatically assess rhythmicity without requiring active user engagement with the user device 110, which considerably improves the impact on clinical care of the patient user. Moreover, passive monitoring performed by the user device 110 can result in obtaining granular and wide-ranging data otherwise cumbersome if not impossible from manual or subjective tracking. In some implementations, for example, the system 100 can provide interventions, such as enabling preemptive care at the right moment and the right place for the subject using the system. For example, by lowering user burden through automated, passive data detection and quantitative assessment, the system 100 can provide crucial and subtle clues that inform clinical decisions on individualized treatment course.

FIG. 1B shows a diagram of an example embodiment of a method 150 for passively monitoring mental health of a subject using a mobile communications device such as a smartphone. The method 150 includes a process 152 to sense a plurality of parameters including location, movement, and sound using the user device 110 associated with the subject. The method 150 includes a process 154 to produce a set of quantitative metrics associated with one or more of the sensed parameters. In some embodiments of the process 154, the set of quantitative metrics includes a location cluster value, a travel distance value, a frequency of conversation value, and an activity value. For example, each of the quantitative metrics is based on the respective sensed parameter over its own respective predetermined time period. In some implementations, the respective time period can be the same for some or all of the quantitative metrics, e.g., such as one day. The respective time period of each quantitative metric can be a contiguous time period or discrete and/or discontinuous time periods over a time frame. For example, the respective time period associated with a quantitative metric can include a morning period, an afternoon period, and an evening period of various lengths for a single day or over a plurality of days, such as a week.

The method 150 includes a process 156 to determine a metric indicative of a clinical state of the subject, the metric in connection with one or more measures including daily routines, mood and/or energy of the subject, e.g., a social rhythm metric. For example, the determined metric indicative of the current clinical state can include a metric associated with social rhythm measures of the subject, such as the SRM (e.g., SRM score). In implementations of the method 150, the SRM is determined at least partly without active input from the subject, where in some implementations the SRM is determined without any active input by the subject using the user device 110. In some implementations, the determined SRM corresponds to a current clinical state of the subject, such as a current mental state for a patient managing a mental health condition like bipolar disorder. In some implementations, the determined SRM corresponds to a future clinical state of the subject, and as such, the determined SRM is a predictive measure.

In some embodiments of the process 154, the set of quantitative metrics includes differing combinations of the individual quantitative metrics. For example, in some embodiments, the set of quantitative metrics includes the travel distance value, the frequency of conversation value, and the activity value. In some embodiments of the process 154, the set of quantitative metrics includes additional quantitative metrics or substitutive quantitative metrics, such as a sleep activity value and/or a device usage value. For example, the device usage value can be produced based on passive monitoring and analysis of the patient user's usage data including but not limited to app usage time, battery usage time or percent, duration or frequency of battery charging status, quantity or frequency of notifications received, quantity or duration of phone calls from a phone log, quantity or frequency of messages received, and/or quantity or frequency of messages transmitted.

In some embodiments of the process 156, determination of the SRM includes applying a machine-learning framework including a support vector regression process to the set of quantitative metrics. For example, the support vector regression process can be applied over a predetermined time frame including a rolling time window of at least seven days. In some embodiments of the process 156, the determined SRM is set on a fixed scale.

In some implementations, for example, the method 150 further includes a process to produce a binary SRM output indicative of the clinical state of the subject, e.g., as being stable or unstable. This process includes comparing the determined SRM corresponding to the clinical state of the subject to a binary threshold SRM defining a range of stability and instability. For example, the quantitative classifiers produced based on the collected streams of sensor data can be processed to generate a SRM on a fixed scale (e.g., SRM score from 0 to 7), in which a binary delineation of the fixed scale is used to predict stable (e.g., SRM score≥3.5) and unstable (e.g., SRM score<3.5).

In some implementations, for example, the method 150 further includes a process to determine a level of change of social rhythmicy of the subject's clinical state. This process includes calculating a differential value between the determined SRM corresponding to the clinical state of the subject and a SRM threshold, and then evaluating the differential value to determine the level of change of social rhythmicy of the subject's clinical state.

In some implementations, for example, the method 150 further includes a process to rank the quantitative metrics to assess the importance of each metric in predicting the determined SRM, e.g., the SRM stability, from the sensed parameters that were passively detected from the user device 110. In some implementations, for example, the process to rank the quantitative metrics includes applying a recursive feature elimination (RFE) model, in which the RFE model is trained on an entire dataset of the sensed parameters to eliminate one quantitative metric that contributes the least to the model, such that this is repeated until a ranking value is produced for each of the quantitative metrics.

In some implementations, the method 150 includes the processes 154 and the processes 156 to produce a set of quantitative metrics based on parameters including location, movement, and sound obtained from the user device 110 associated with the subject, in which the produced set of quantitative metrics includes a location cluster value, a travel distance value, a frequency of conversation value, and an activity value, and each of the quantitative metrics is over a respective predetermined time period; and to process the set of quantitative metrics to determine the metric indicative of a current clinical state of the subject in connection with one or more measures including daily routines, mood and/or energy of the subject, e.g., the SRM.

FIG. 1C shows a flowchart representation of another example embodiment of a method 170 for passively monitoring and assessing a current mental state or condition of a user (e.g., patient user) using the system 100, e.g., implemented by the user device 110 such as a smartphone equipped with the app 120. The method 170 includes, at 172, sensing at least one physical parameter related to the patient user's ambient environment. In some implementations, the physical parameter includes one or more of the patient user's location, sounds heard by the patient user, and/or ambient light near the patient user. For example, in some embodiments, the sensing the physical parameter includes sensing the patient's movement at various times of day. The method 170 includes, at 174, tracking usage data of the patient user's use of the user device 110. In some embodiments, the usage data includes the patient user's call log and/or text messaging usage. The method 170 includes, at 176, estimating, at least partly without explicit input from the patient, the patient user's current state or condition from the physical parameter and the usage data. In some embodiments, the method 170 may further include, based on the sensed physical parameter including the patient user's movement, estimating a state of the patient's circadian rhythm.

FIG. 3 shows a diagram of an example software architecture 300 for processing passively monitored data and quantitatively characterizing social rhythms (e.g., SRM) of a user in accordance with embodiments of the present technology. In some embodiments, the software architecture 300 is embodied as part of the software application 120 on the user device 110. Similarly, in some embodiments, for example, the software architecture 300 can be embodied as part of the data processing system 130, e.g., on the cloud computer(s). In some embodiments, the software architecture 300 is embodied as part of both the software app 120 on the user device 110 and the data processing system 130, e.g., in which some or all of the modules of the software architecture 300 are shared or divided among the app 120 and the data processing system 130.

The software architecture 300 includes a data collection module 310, a SRM processing module 320, a device unit controller module 330, and a SRM standard module 340. In implementations, for example, the data collection module 310 aggregates data from the sensor units of the user device 110, e.g., ambient light sensor, camera, microphone, accelerometer, etc., input units of the user device 110 (e.g., buttons, display, etc.), and/or functional units of the user device 110 (e.g., GPS, software applications, etc.). For example, data obtained by the data collection module 310 can include motion data, light data, sound data, GPS data, device usage data, and/or user input data. The SRM processing module 320 processes the collected data to produce a SRM score based on the collected data. In some implementations, the data collection module 310 may pre-process the collected data to generate organized groups of the data, quantitative metrics associated with the data, or other. In some implementations, the SRM processing module 320 processes the collected data to generate organized groups of the data, quantitative metrics associated with the data, or other. In some implementations, the SRM processing module 320 can determine the SRM based on predetermined thresholds or variable thresholds, e.g., such as a binary threshold SRM to define a range of stability or instability of a determined SRM. The device unit controller module 330 can provide a control signal or control data set to various device units of the user device 110 (e.g., camera, display, speaker, microphone, etc.) to control a functionality of the device unit based on a prompt from the data collection module 310 and/or the SRM processing module 320.

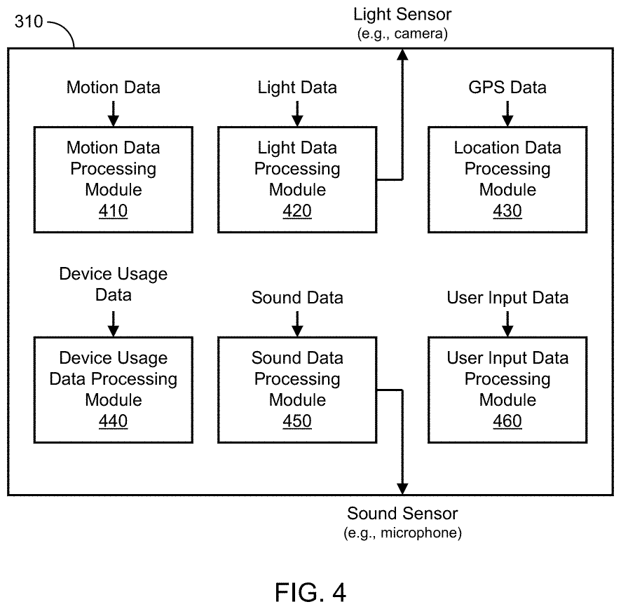

FIG. 4 shows a diagram of an example data collection module 310 of the software application in accordance with embodiments of the present technology. The data collection module 310 includes a motion data processing module 410 to obtain and process motion data acquired from motion sensor 113 of the user device 110. The data collection module 310 includes a light data processing module 420 to obtain and process data associated with light or images acquired from the image sensor 111 of the user device 110. The data collection module 310 includes a location data processing module 430 to obtain and process GPS data from the user device 110. The data collection module 310 includes a device usage data processing module 440 to obtain and process usage data acquired by various functional units of the user device 110. The data collection module 310 includes a sound data processing module 450 to obtain and process sound data acquired from the sound sensor 112 of the user device 110. The data collection module 310 includes a user input data processing module 460 to obtain and process input data entered by the user acquired by various functional units of the user device 110.

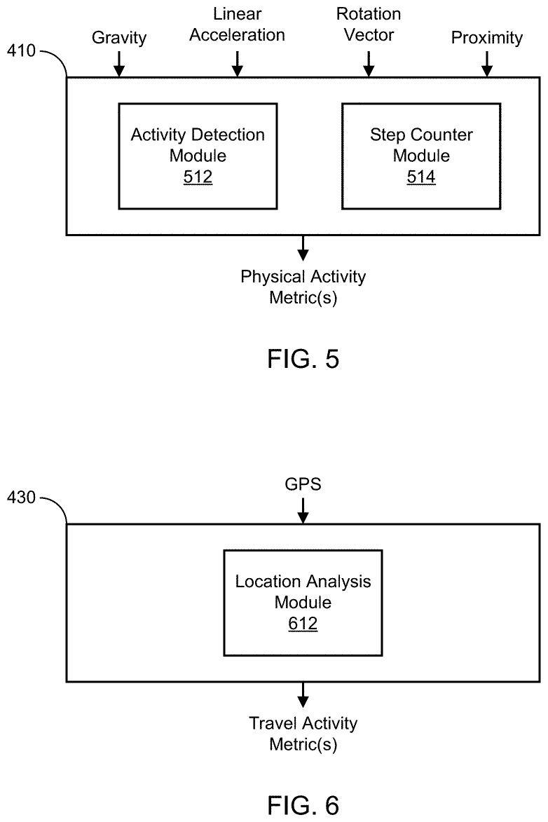

FIG. 5 shows a diagram of an example embodiment of the motion data processing module 410. The motion data processing module 410 can include an activity detection module 512 and a step counter module 514. In some implementations, motion data including one or more of gravity, linear acceleration, rotation vector and proximity data detected by a motion sensor or sensors of the user device 110 is received by the activity detection module 512, which determines one or more metrics associated with the movement-based activity of the user device 110, and thereby a measured physical activity of the patient user, e.g., over a particular time period such as each day or a predetermined time frame. In some implementations, the activity detection module 512 utilizes software modules (e.g., program code as part of the operating system of the user device 110) to calculate the one or more activity metrics. For example, the activity detection module 512 can quantify activity of the patient user when he/she is walking, running, biking, etc. versus when he/she is riding in a vehicle or is stationary. In some implementations, motion data including one or more of gravity, linear acceleration, rotation vector and proximity data detected by a motion sensor or sensors of the user device 110 is received by the step counter module 514 to determine a total number of steps taken by the patient user using the user device 110 over a predetermined time period, e.g., steps taken by the user each day, within a certain time frame such as morning, afternoon, evening, etc., or a certain time frame after a certain time or event, or the like.

FIG. 6 shows a diagram of an example embodiment of the location data processing module 430. The location data processing module 430 can include a location analysis module 612 that receives GPS data and determines metrics associated with the patient user's travel activity or venue change behavior. For example, the location analysis module 612 can use the GPS data to calculate a location cluster value using a location density algorithm that clusters values of the patient user's location via the sensed location (e.g., GPS data) from the user device 110, e.g., over a certain radius from a default location. For example, the radius can include 0.5 km, or more or less, from the patient user's home, place of work, etc. In some implementations, the location analysis module 612 can calculate total distance traveled by the patient user over a predetermined time period, e.g., each day and/or within a certain time frame. Similarly, for example, the location analysis module 612 can use the GPS data to calculate the number of different location clusters traveled for each day. In some implementations, the location analysis module 612 can apply a density-based algorithm with a predetermined radius from a given home location (e.g., radius of 0.5 km from the user's residence) to identify these clusters. The home location can be modified to accommodate a user's travels, such as when the user may have moved or is temporarily staying in another location. Each of these location clusters can be used to point to significant places for a given participant (e.g., home, work places). As such, the location analysis module 612 can determine visiting patterns over these location clusters which can provide information about regularity in daily routine for the patient user.

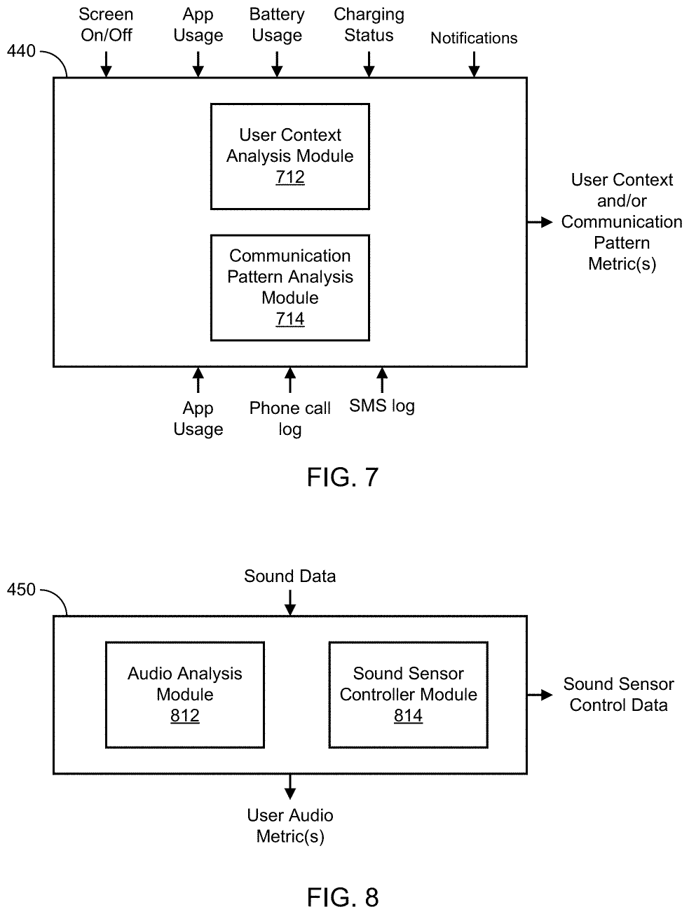

FIG. 7 shows a diagram of an example embodiment of the device usage data processing module 440. The device usage data processing module 440 can include a user context analysis module 712 that receives usage data of the user device 110 and determines one or more user context metrics associated with the patient user's environment, relationships and/or behavior with respect to his/her usage of the user device 110. For example, the user device 110 usage data can include screen operation data (e.g., screen on/off), which is indicative of how much and/or how frequently the patient user operates the user device 110. The usage data can include software application usage data (e.g., how many and how long an app is open in the foreground or background modes of the user device 110, types of application used such as productivity, social media and/or entertainment apps, and/or context switch information), which is indicative of how the patient user uses the user device 110 and thereby suggestive or revealing of his/her social behavior. The usage data can include notification data, such as log data about quantity and timing of notifications from various apps, e.g., including calendar notifications, text message notifications, phone call notifications, news notifications, travel information notifications, and the like. The usage data can include battery usage and charging status data, i.e., if the phone is connected to a charger or not, as such information can be indicative about the duration of total phone usage, duration of each session (e.g., if a session is a short one or long one), and the like.

The device usage data processing module 440 can include a communication pattern analysis module 714 to generate pattern metrics regarding how the user device 110 is used to communicate with other user devices, e.g., including patterns among phone calls, SMS, or communications via apps. The communication pattern analysis module 714 receives communication log information such as SMS logs and call logs, etc. In some implementations, for example, the operating system of the user device 110 keeps records of such information, which the communication pattern analysis module 714 can query to obtain the information. The communication pattern metrics can be analyzed with the user context metrics to produce a data set of multiple metrics or a single metric.

FIG. 8 shows a diagram of an example embodiment of the sound data processing module 450. The sound data processing module 450 can include an audio analysis module 812 and a sound sensor controller module 814. The audio analysis module 812 is configured to process the sound data obtained by the sound sensor 112 to determine if it is human speech related to the patient user, e.g., as opposed to background voices or non-live voices from media such as television, radio, etc. In some implementations, the sound sensor controller module 814 provides a control signal or data set to cause the sound sensor to become activated and deactivated to selectively capture the ambient sound for analysis by the audio analysis module 812. For example, the sound sensor 112 of the user device 110 can be activated on a periodic basis (e.g., every minute, 2 minutes, or other predetermined interval) to capture the ambient sound in the environment of the user device 110. If the audio analysis module 812 determines the captured ambient sound is human speech, the sound sensor controller module 814 generates control data to keep the sound sensor 112 (e.g., microphone of a smartphone) active. The audio analysis module 812 includes algorithms to determine relevant voice data to the patient user and filter out false positives, such as conversation from TV and radio programs, e.g., by energy intensity and distribution likelihood. In some implementations, for example, the audio analysis module 812 is configured to not record the ambient sound detected by the sound sensor 112, but rather analyze the obtained audio data in real-time to only extract and store analyzed features, e.g., spectral content, regularity, volume and pitch magnitudes and changes. In this manner, the audio analysis module 812 can protect privacy concerns of the user by detecting the presence of human voice insufficient to analyze content in speech. The audio analysis module 812 produces user audio metrics that are indicative of social rhythms, such as the number of conversations the patient user engages in, how long the conversations last, and his/her activity during the conversations including speaking rate and tone variations.

In some implementations, the sound data processing module 450 can determine a frequency of conversation involving the patient user using the sensed sound obtained by the sound sensor 112. For example, the sound data processing module 450 can control the sound sensor 112 to sample audio over a time period. For example, the sound data processing module 450 can intermittently or periodically sample the audio signals detectable by the sound sensor 112. The sound data processing module 450 can process the sampled audio to determine speech from other sound present in the sampled audio, and from that discern live human speech from non-live voices present in the sampled audio (e.g., such as live conversation from that of TV, radio, or other non-live conversations). In determining the frequency, duration, or other metric associated with conversation, the sound data processing module 450 can samples the audio in a manner that does not include recording speech, such that the determination of live human speech includes analyzing the sampled audio in real time including extracting one or more audio features (e.g., spectral content, regularity, volume, and/or pitch magnitudes and changes) without creating privacy concerns of the patient user or others around him/her.

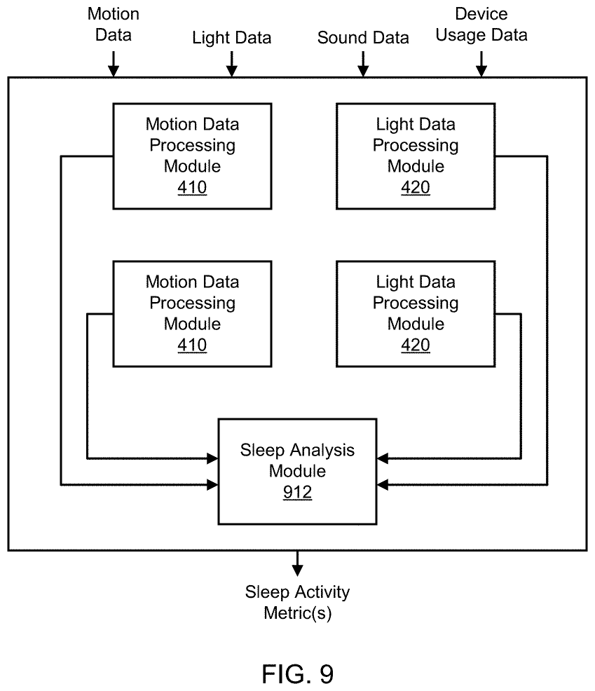

FIG. 9 shows a diagram of an example embodiment of a sleep analysis module 912, which can be included as a module of the data collection module 310 and/or the SRM processing module 320. In some implementations, the sleep analysis module 450 receives processed metrics associated with the motion data, light data, sound data and/or device usage data to determine sleep activity metric(s). For example, sleep can be a really important feature in detecting relapse onset in bipolar. In implementations, for example, the sleep analysis module 450 can automatically and passively sense and quantitatively assess instances and aspects of sleep by the patient user using the user device 110, e.g., based on sleep onset and duration. For example, the sleep analysis module 912 can process sensed light data with at least one of the sensed motion data or device usage data to produce a sleep activity value to be included in the set of quantitative metrics at the process 154 of the method 150. In some implementations, the sleep activity value can be used to determine sleep-wake rhythms and/or light-dark exposure that affect various clinical conditions including bipolar disorder.

Example Implementations

Described below are implementations of example embodiments of the systems, methods and devices to passively monitor and assess social rhythms in bipolar disorder. In the example study, the feasibility of automatically assessing the SRM using an example embodiment of the system was evaluated. In the study, seven patients with bipolar disorder used their smartphones that included an example embodiment of the app 120 for four weeks, which passively collected and processed sensor data including accelerometer, microphone, location and communication information to infer behavioral and contextual patterns. As a control, for example, the participants also completed manual entries associated with the SRM using a smartphone app. The example results indicated that automated monitoring and assessment system successfully determined the SRM score with respect to the control method to determine the SRM for the respective patients. For example, using location, distance travelled, conversation frequency and non-stationary duration as inputs, the system model achieved root-mean-square-error (RMSE) of 1.40, e.g., which was a reasonable performance given the range of SRM score (0-7). Personalized models of the system further improve the comparative performance with mean RMSE of 0.92 across users. Furthermore, for example, classifiers using sensor streams were used to predict stable (e.g., SRM score≥3.5) and unstable (e.g., SRM score<3.5) states with high accuracy (e.g., precision: 0.85 and recall: 0.86).

In the study, the use of smartphone-based sensing to overcome the limitations of existing self-report methods were focused on to help patients with confirmed diagnosis of bipolar disorder maintain stability and rhythmicity. To achieve this goal, an example embodiment of the smartphone app 120 was employed on the patients' smartphone devices, which passively collected behavioral data (e.g., speech, activity, SMS and call log) and contextual data (e.g., location) based on smartphone sensors for four weeks during the study. Based on the collected data, machine learning techniques of the system 100 were used to model and predict markers of rhythmicity in the daily life of the patients with bipolar disorder, e.g., to reduce the risk of relapse. The system was employed to assess a variety of indicators of stability and rhythmicity that might have impact on social rhythms and automatically infer the stability and rhythmicity based on SRM scores using the passively collected sensor data. Since social rhythms are central to the wellbeing of individuals with bipolar disorder, successfully measuring social rhythms via passive smartphone-based measures can have considerable potential for the widespread, successful use of monitoring and treatment of bipolar disorder.

Potential participants for the example study were identified and sent information letters to solicit participation. For example, these letters stated project goals, expected duties, and time commitment. Patients interested in learning more were referred directly to the research staff who provided more information about the study and obtained informed consent from those interested in participating. Seven patients were ultimately selected for and participated in the study.

After consent administration, enrolled participants completed an initial questionnaire. The participants were provided a customized smartphone app, which included features of the app 120 for passive monitoring and assessment of social rhythms, and features to allow manual entry in accordance with the SRM standards, e.g., for comparative control purposes. The participants were taught how to use the app and participate in the study. The study lasted 4 weeks, and the participants completed a post-study questionnaire and interview when it was completed.

The customized smartphone app was designed to track the user's social rhythms and supported both subjective rating (e.g., manual data collection) and automatically-sensed data collection. The customized smartphone app allowed patients to track five core activities used in a pen-and-paper version of the SRM-5, which included (1) waking time, (2) first contact with another individual, (3) starting their day, (4) dinner, and (5) bedtime. The customized smartphone app also included custom activities, e.g., such as allowing the user to track his/her mood and energy on scale of −5 (very low) to +5 (very high).

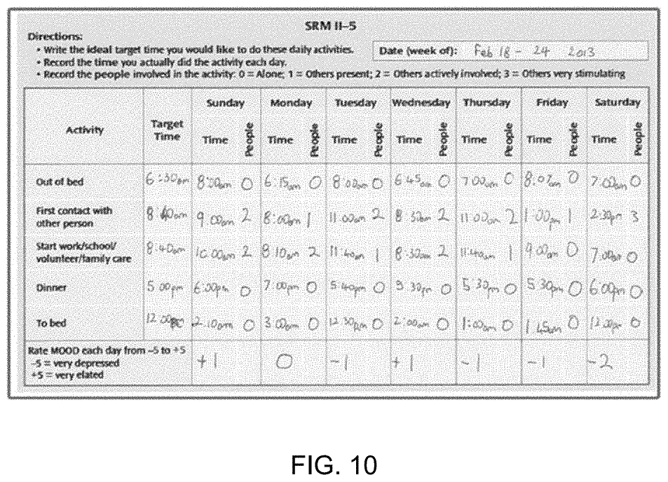

FIG. 10 shows an example of a paper-based Social Rhythm Metric (SRM) form that is used as part of Interpersonal Social Rhythm Therapy (ISRT) process.

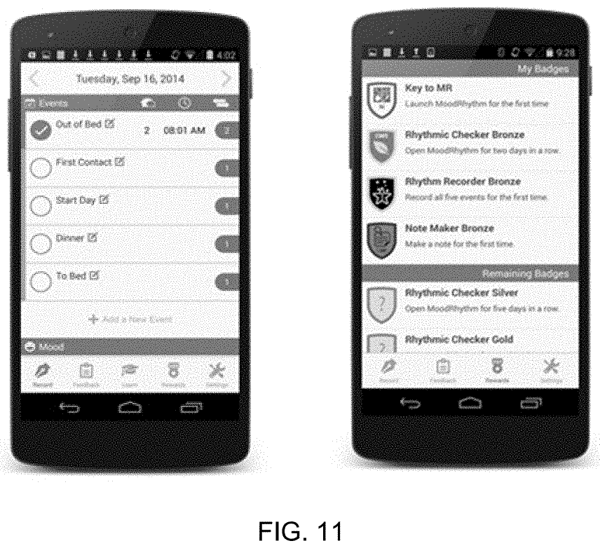

FIG. 11 shows screenshots of an example user interface of the customized smartphone app used in the example study. The example screenshot (left) shows a daily checklist that the participant was instructed to complete if/when they did the following actions, e.g., get out of bed (noting the time), had a first contact with another person, begin their day, have a meal (e.g., dinner), and go to bed. The example screenshot (right) shows badges or other rewards for carrying out various actions associated with the study.

The patient participants were instructed to set daily target times for activities and track how closely they meet these target times. Notes can be used to record additional information such as the amount of medication taken or factors that may have affected a patient's routine or mood. The customized smartphone app included an at-a-glance summary of the person's successes in meeting their rhythm goals for both the current and preceding days. For example, if the patient completed an activity within their customizable time window (e.g., the default is 45 minutes), then the bar to the left would turn green. When the window is about to elapse and an event is not yet recorded, the bar appeared yellow (e.g., a “warning” that a potential rhythm disruption is occurring). If a target is missed, then the bar turned red. More detailed feedback on weekly patterns was viewable in weekly graphs. Also, for example, the customized smartphone app incorporated a series of badges that are given based on adherence to self-report.

The example smartphone app implemented in the study used a variety of sensor data sources on the smartphone platform with the ultimate aim of inferring behavioral rhythmicity. The example platform continuously collected data from the phone's light sensor, accelerometers, and microphone, as well as communication patterns and information about phone usage events such as screen unlocks and charging. For example, the smartphone microphone was activated every 2 minutes to capture ambient sound. If human speech was detected, the microphone remained active. To filter out false positives including conversation from TV program, for example, energy intensity and distribution likelihood was used. The user's privacy was respected and protected, as the example smartphone app did not record audio recordings, but instead processes data in real-time to only extract and store features (e.g., spectral content and regularity, loudness) that are useful for detecting the presence of human voice but insufficient to reconstruct speech content. Using these privacy-sensitive audio features and probabilistic inference techniques, it is possible to reliably estimate the number of conversations an individual engages in, the duration of the conversations, and how much time a given individual speaks within a conversation along with speaking rate and variations in pitch. Such features of the example smartphone app can be used to detect social isolation in users, e.g., such as older adults.

Activity was captured by the smartphone accelerometer that detects movement. The system generates and stores physical activity status (e.g., active vs. sedentary). For location detection, for example, the smartphone's location services features were employed, which combined Global Position System (GPS), Wi-Fi and cellular data to provide location estimations. The example smartphone app also collected communication patterns including SMS and call logs. The sensor data were stored on the smartphone and securely transmitted to the remote server(s) of the system, e.g., periodically. The impact of the example smartphone app on battery was also assessed and determined to be reasonable in its power use to implement the passive monitoring and assessment of social rhythms in the example bipolar disorder study, e.g., permitting 16 hours of continuous sensing after a full recharge.

Example results of the study included the following. On average, participants recorded 36.5 (SD: 11.17) energy instances, 46.12 (SD: 12.71) mood instances and 144.43 (SD: 43.1) SRM event entries including custom activities. From sensor data, the overall distance travelled per day by each participant is 8.34 km (SD: 13.34). On average, the ratio of time being sedentary to active per day is 2.09. Participants were around human speech 3.25 hours (SD: 3.67) a day, on average. The trend of self-assessed energy scores—computed over seven days (rolling average), which correlated with sensor streams as shown in Table 1. Mood pattern is weakly correlated with conversation (r=0.16, p=0.06) and non-sedentary duration (r=0.15, p=0.08).

TABLE 1
Correlation between sensor stream and trend of self-
assessed energy scores computed as rolling average
over 7 days (**: p < 0.01, ***: p < 0.001).

| Sensor | Correlation |
|---|---|
| Cluster | 0.31*** |
| Distance | 0.23** |
| Conversation | 0.25** |
| Non-sedentary duration | 0.39*** |

The example study focused on automatic inference of rhythmicity in the daily life of patients with bipolar disorder using an example embodiment of the system 100. The sensed data was used to build a statistical inference and prediction model using machine learning techniques in accordance with the present technology. Patient-reported data to generate the SRM score as used as a comparative control. The system employed a predictive model to infer the SRM score from smartphone-based sensor data alone, e.g., without patient data entry or conscious participation. In particular, the example predictive model used the number of location clusters, distance traveled, frequency of conversation determined from processing the sensed audio data, and duration of non-sedentary activity calculated over each day as inputs to the model (e.g., feature set). For location clustering, the example predictive model used a density based algorithm, e.g., with a radius of 0.5 km. The example key features were selected because they are good indicators of social and physical functioning, which are key in tracking symptomatic behavior of patients with mental health conditions like bipolar disorder. For example, speech and conversation features can be used for determining states of individuals with bipolar disorder, and levels of physical activity, location and mobility can indicate state change in this disorder. Moreover, as shown in Table 1, these features correlate with self-reported energy.

The example system used in the study was capable of calculating SRM scores even with the high granularity of sensed data, where SRM scores were calculated using a rolling window of 7 days. It is noted that conventional means for determining SRM scores have been limited to non-overlapping weeks, e.g., due to limitations previously discussed. In the study, the value of the SRM score ranged from a theoretical “0” to a theoretical “7” where higher values indicate greater rhythmicity. SRM scores were in a continuous range, so to model them a Support Vector regression was used, e.g., a machine learning framework. The accuracy of the model was evaluated using 10-fold cross-validation, e.g., a modeling validation technique in statistical analysis. For this, data was randomly partitioned into 10 equal sized subsets. For each round, for example, a single subset was retained for model validation and the other 9 subsets were used for training the model. This process was repeated 10 times to use each subset for validation and the averaged result was then computed. Using test set independent of training data can help assess the generalizability of model. From the example analysis, it was found that the average root mean square error (RMSE) was 1.40. Given the range of SRM scores (0 to 7), the low RMSE value indicates that the model achieved reasonably good accuracy. Personalized models trained on each individual can significantly improve performance with a mean RMSE of 0.92 across all participants. It is noted, for example, that model performance would likely improve further with the collection of more data enabling better adaptation to idiosyncratic trends over longer period of time.

Beyond raw SRM scores, the ability to infer status of rhythmicity from sensor data was also investigated. For example, from a large study on representative healthy population (n=1249), it was found that the mean population SRM score is close to 3.5. Following this finding of a “normal social rhythm”, it was considered for this study that a SRM score lower than 3.5 was an unstable state, while any score greater or equal to 3.5 was indicative of a stable state. In this example formulation, a binary (e.g., unstable and stable) classification problem can be formed. Support Vector Machine (SVM) was used for prediction. Based on input features, SVM constructs decision boundary separating different classes (e.g., stable and non-stable states in this case). For training the model, the same feature set was used as before. Over 10-fold cross-validation, the example model achieved high performance with a precision score of 0.85 and recall score of 0.86.

Feature ranking was also performed to assess the importance of each feature in predicting SRM stability from sensor data. Recursive feature elimination (RFE) was used for this, for example. At each step of RFE, a model is trained on an entire dataset and the feature least contributing to the model is discarded. This procedure is recursively continued until there is only one feature left. The most important features for prediction in this example were the location cluster and total distance travelled over a day, as shown in Table 2.

TABLE 2
Ranking of feature importance for the stable and unstable
status classification using recursive feature elimination
(RFE). Table 2 also shows weights assigned to features
in a support vector machine using linear kernel.

| Feature | Ranking | Weight |
|---|---|---|
| Distance Travelled | 1^{st} | 1.56 × 10⁻² |
| Location Cluster | 2^{nd} | 3.27 × 10⁻³ |
| Non sedentary duration | 3^{rd} | −3.79 × 10⁻⁴ |
| Conversation frequency | 4^{th} | 7.69 × 10⁻⁵ |

A class probability estimate was computed that provided more granular information than prediction output alone. Probability estimations are easily interpretable and particularly useful in conveying uncertainty associated with outputs from a statistical model. In particular, the confidence score associated with predictions made by the model could help clinicians make informed decisions. For example, depending on the potential cost associated with misclassification in a given context, clinicians could choose to discard predictions with high uncertainty. For example, being able to associate a confidence score with this binary outcome can be particularly useful in clinical setup, e.g., where if the prediction has low confidence score (e.g., with probability 0.51—barely more than a threshold), then it might require further reviews from the clinicians.

To calculate the probability distribution from the outputs of our classification model, Platt scaling was used. To calculate probability, it fits a logistic regression model to the classifier output. From the calculated probability estimates, it was found that 75.89% of correctly classified labels have a probability≥0.7, e.g., an indicator of high confidence in predictions from the learning model. In other words, for the majority of the correctly classified labels, the learning model has high confidence. For example, this showed that the example prediction model is quite robust against noise.

This example study, it is believed, is the first study that automatically infers stability and rhythmicity as assessed from the SRM score using passive sensor data. The example system embodiment employed a predictive model that can distinguish between stable and unstable states with high precision. The probability estimation implemented in the study also indicated that the model is quite robust. The implications of these and other findings are discussed below, e.g., such as operating the system as an early warning system to predict relapses. The ability to automatically detect departures from rhythmicity as described here can open up ways of providing instantaneous interventions beyond the current capabilities of existing clinical systems.

Managing bipolar disorder requires constant and lifelong vigilance against relapse. Interventions targeting the regularity of social rhythms can lower the risk of relapse and improve long-term prognosis. However, maintaining self-tracking over a long period of time using the existing paper-and-pencil SRM is understandably challenging. For example, patients can forget to complete entries, and there is no way to recover lost records. More importantly, relying on the individual patient's ability to recall events, e.g., whether the self-report measure is administered on a paper form or even on a smartphone, often fails to capture the subtler details of behavioral and contextual patterns that may be particularly difficult to track for people with severe psychiatric disorders.

The systems, methods and devices in accordance with the present technology provide a viable, robust and effective platform for passively and automatically monitoring social rhythms and measuring one's stability or instability in managing a mental illness. As shown by the findings in the example study, the example system demonstrated viability of overcoming the limitations of existing clinical tools. For example, the example predictive model did not rely on individual recall of events, and thereby removes the risk of non-adherence and stigma. The wide ranging data collection capabilities combined with the unobtrusive nature of smartphone-based sensors means that behavioral and contextual tracking of daily patterns can be much more comprehensive and continuous. Notably, the original SRM scale had 17 items, but was reduced to 5 items because of the difficulty in manually tracking so many items. Yet, smart device-based automatic sensing systems in accordance with the disclosed technology can track a wide array of trends, without placing additional burden on users, and therefore possess the potential to identify individualized cues that might be more accurate idiosyncratic markers of clinical status than the 5 self-rating items in conventional SRM techniques. Also, for example, over long periods of time, data provided by systems in accordance with the disclosed technology may also be helpful in identifying person-specific disruptors of routines and prodromes of episode onset.

Automatic and unobtrusive sensing of rhythmicity provided by systems in accordance with the disclosed technology can also address issues with longitudinal data tracking. For example, in contrast to paper-and-pencil measures, active user self-involvement in recording behaviors and activity is not necessary to track daily trends. This might be particularly useful when the patient is very symptomatic, e.g., remembering to complete SRM items is understandably more unlikely during the depths of depression and the heights of mania, and this is precisely the time when symptom tracking is most crucial. Thus, automated data collection and trend detection before relapse onset can provide invaluable insights for immediate intervention and clinical actions for the long run. For example, a low conversation level for an extended period of time might be indicative of the onset of a depressive state while increased number of location clusters and distance travelled might signal the beginning of a manic phase. Such early detection of relapse signatures can enable preemptive care.

Another problem with how existing tools are used in practice is the lag in intervention. For example, the delay between recording SRM entries and clinicians having access to them can be longer than ideal. Being able to automatically assess stability and rhythmicity can help in the provision of more timely feedback to individuals outside of clinical settings by identifying and sharing disruptions in routines in real time. Beyond just a tracking tool, smartphones can also be an intervention delivery tool, e.g., providing care to patients when and where they need it.

Moreover, the data provided by the passive sensing techniques of the present technology that obtain and analyze contextual and behavioral trends for longitudinal wide-ranging tracking can enable a better understanding of individualized symptom cues for both patients and clinicians. The automated sensing and prediction techniques in accordance with the present technology are thus envisioned to empower patients and also enable clinicians to create more effective personalized treatment plans. In an illustrative example, clinicians could combine the output from sensed data with other subjective measurements. Along with classifier decisions, the confidence score from the probability estimates could also help clinicians to make informed decisions about the uncertainty.

Furthermore, in some embodiments, the system can employ the self-reporting techniques with the automatic and unobtrusive sensing and analysis techniques of rhythmicity in accordance with the disclosed technology. For example, by combining the self-reported SRM with automated, passive sensing by the smartphone for a short period of time, the models can further be individualized to each patient. This may further improve the accuracy of clinical information. Since bipolar disorder is a life-long condition characterized by common and idiosyncratic symptoms, this training period could be very helpful.

Although passive smartphone sensing of social rhythms based on our generalized model can provide valuable and otherwise-unobtainable clinical information, it is important to consider whether any positive therapeutic elements might be lost by using a fully automated system in clinical practice. For example, in a sensor-supported system, there is a risk that positive elements associated with self-tracking such as having a sense of involvement in treatment and control over one's illness may be lost. This is particularly relevant in the case of the SRM which is both a measure of social rhythms and a tool for helping individuals structure their days by explicitly setting target times for each event. While the disclosed approach enables clinicians to not have to rely entirely on self-reported measures of social rhythmicity, participants may still continue to self-track if deemed therapeutically beneficial. In some example embodiments of the system, self-reporting techniques are included with the automatic and unobtrusive sensing and analysis techniques of rhythmicity. For example, in such embodiments, it is possible to qualitative aspects of an individual experience in managing their condition can be assessed.

Since maintaining stability in daily routine can significantly reduce risk of relapse in individuals with bipolar disorder, being able to automatically assess rhythmicity without requiring active user engagement can have considerable impact on clinical care. In particular, the example results and findings from the example study indicate that embodiments of systems and methods in accordance with the present technology can be used to help overcome issues with existing paper-and-pencil based clinical tools, e.g., such as by significantly lowering user burden of manual tracking. As passive sensing can result in much more granular and wide-ranging data than manual and subjective tracking, these example results can be extended to an early warning system for relapse detection. Such a system can provide interventions, such as enabling preemptive care at the right moment and the right place for the subject using the system. For example, by lowering user burden, the use of automated sensing could make longitudinal tracking significantly easier, which, in turn, can provide crucial and subtle clues to inform clinical decisions on individualized treatment course. In addition to bipolar disorder, for example, the SRM has also been used in a number of clinical conditions including stroke, Parkinson's disease, myoclonic epilepsy, anxiety disorders and unipolar depression. As such, it is envisioned that the systems, methods and devices in accordance with the present technology can be used to automatically infer stability and rhythmicity in applications for these conditions as well, e.g., which is perceived to greatly enhance the practicality of social rhythm theory as a clinical tool and research instrument.

For example, circadian disruption has been linked to other neuropsychiatric illnesses, e.g., including depression and schizophrenia. As the Earth rotates around its axis approximately every 24 hours, most organisms are subjected to periodic changes in light and temperature resulting from exposure to the Sun. Given the constancy of this phenomenon over the course of evolution, nearly every living creature has developed internal biological clocks to anticipate these geophysical fluctuations. These biological clocks drive our “circadian” system. “Circadian” means about (circa) a day (diem), and our circadian rhythms reflect any biological cycle that follows a roughly 24-hour period such as regular changes in our blood pressure, cortisol, and melatonin levels.

Circadian system plays a crucial role in synchronizing our internal processes with each other and with external environments. However, a number of factors can disrupt an individual's circadian systems and, in turn, sleep-wake cycles, mood, and, levels and timing of hormone secretions. These symptoms have been associated with a wide range of mental health problems including alcohol and substance abuse, anxiety, attention-deficit hyperactivity disorder, bipolar disorder, depressive disorder, obsessive-compulsive disorder, and schizophrenia.

As such, the disclosed systems, methods and devices for automatically assessing stability and rhythmicity are envisioned to be implemented to enable clinical decision making and preemptive care for a wide range of mental health care, such as these mental health conditions affected by circadian-related inputs. Moreover, the disclosed systems, methods and devices are envisioned for use in circadian-related applications beyond the context of mental health.

### EXAMPLES

In some embodiments in accordance with the present technology (example A1), a method for passively monitoring a health condition of a subject using a mobile device includes producing a set of quantitative metrics based on one or more parameters including location, movement, and sound obtained from a mobile communications device associated with the subject, in which the produced set of quantitative metrics includes a location cluster value, a travel distance value, a frequency of conversation value, and an activity value, and each of the quantitative metrics is over a respective predetermined time period; and processing the set of quantitative metrics to determine a metric indicative of a current clinical state of the subject in connection with one or more measures including daily routines, mood and/or energy of the subject. For example, the determined metric indicative of the current clinical state can include a metric associated with social rhythm measures of the subject, such as the SRM.

Example A2 includes the method of example A1, in which the metric indicative of the current clinical state is determined at least partly without active input from the subject.

Example A3 includes the method of example A1, in which the processing includes applying a machine-learning framework including a support vector regression process to the set of quantitative metrics.

Example A4 includes the method of example A3, in which the support vector regression process is applied over a predetermined time frame including a rolling time window of at least seven days.

Example A5 includes the method of example A1, in which the determined metric indicative of the current clinical state is set on a fixed scale.

Example A6 includes the method of example A1, further including comparing the determined metric indicative of the current clinical state of the subject to a binary threshold defining a range of stability and instability; and producing a binary output indicative of the current clinical state of the subject being stable or unstable.

Example A7 includes the method of example A1, further including determining a differential value between the determined metric indicative of the current clinical state of the subject and a threshold; and evaluating the differential value to determine a level of change of social rhythmicy of the subject's current clinical state.

Example A8 includes the method of example A1, further including ranking the quantitative metrics to assess importance of each quantitative metric in predicting stability of the determined metric from the parameters.

Example A9 includes the method of example A8, in which the ranking the quantitative metrics includes applying a recursive feature elimination (RFE) model, in which the RFE model is trained on an entire dataset of the sensed parameters to eliminate one quantitative metric that contributes the least to the model, and is repeated until a ranking value is produced for each of the quantitative metrics.

Example A10 includes the method of example A1, in which the location cluster value is produced using a location density algorithm to cluster values of the subject's location via the sensed location of the mobile communication device over a predetermined radius from a default location value.

Example A11 includes the method of example A10, in which the predetermined radius includes 0.5 km or more from the subject's home.

Example A12 includes the method of example A1, in which the travel distance value is produced using one or both of the location parameter and the movement parameter.

Example A13 includes the method of example A1, in which the activity value includes a non-sedentary duration over the respective predetermined time period.

Example A14 includes the method of example A13, in which the activity value further includes an intensity of the subject's movement associated with a magnitude of the movement exceeding a predetermined threshold over a predetermined duration or at a predetermined frequency over the respective predetermined time period.

Example A15 includes the method of example A1, further including sensing the parameters including the location, the movement, and the sound using a location sensor, a motion sensor, and a sound sensor, respectively, of the mobile communications device. In some implementations of the method of example A15, the sensing includes passively and autonomously sensing the parameters.

Example A16 includes the method of example A1, in which the frequency of conversation is produced using the sensed sound, and the method further includes sampling audio from a sound sensor of the mobile communications device; determining human speech from other sound present in the sampled audio; and discerning live human speech from non-live voices present in the sampled audio.

Example A17 includes the method of example A16, in which the sensing the sound further including controlling the sound sensor to selectively sample the audio on a periodic or intermittent basis.

Example A18 includes the method of example A16, in which the sampling the audio does not include recording speech, in which the determining live human speech includes analyzing the sampled audio in real time including extracting one or more audio features including spectral content, regularity, volume, or pitch magnitudes and changes.

Example A19 includes the method of example A1, in which the predetermined time period for at least one of the quantitative metrics is one day.

Example A20 includes the method of example A1, in which the one or more parameters further includes light, and in which the light is sensed using one or more of an ambient light sensor or camera of the mobile communications device.

Example A21 includes the method of example A20, further including producing a sleep activity value included in the set of quantitative metrics, the sleep activity value associated with the sensed light and the sensed movement.

Example A22 includes the method of example A1, further including tracking usage data of the subject's use of the mobile communication device; and producing a quantitative metric associated with tracked usage data to include with the produced set of quantitative metrics, such that the produced set of quantitative metrics further includes a device usage value.

Example A23 includes the method of example A22, in which the usage data includes one or more of app usage time, battery usage time or percent, duration or frequency of battery charging status, quantity or frequency of notifications received, quantity or duration of phone calls from a phone log, quantity or frequency of messages received, or quantity or frequency of messages transmitted.

Example A24 includes the method of example A1, in which the mobile communication device includes a smartphone, a tablet, a smartwatch, or a smartglasses device.

In some embodiments in accordance with the present technology (example A25), a device for passively monitoring a health condition includes a plurality of sensors including a location sensor, a motion sensor and a sound sensor, in which the sensors detect location data, movement data, and sound data associated with a user of the device; and a data processing unit including a memory to store data from the sensors and a processor configured to processes the location data, the movement data and the sound data to generate a set of quantitative metrics including a location cluster value, a travel distance value, a frequency of conversation value, and an activity value, in which each of the quantitative metrics is over a respective predetermined time period, and to determine a metric indicative of a current clinical state of the user in connection with one or more measures including daily routines, mood or energy of the user, in which the metric indicative of the current clinical state is determined at least partly without active input from the user.

Example A26 includes the device of example A25, in which the device includes a smartphone, a tablet, a smartwatch, or a smartglasses device including a software application comprising program code executable by the processor and stored in the memory to provide instructions for the processor to generate the set of quantitative metrics and determine the metric indicative of the current clinical state.

Example A27 includes the device of example A25, in which the plurality of sensors further includes one or more of an ambient light sensor or a camera to detect light data, and in which the set of quantitative metrics further includes a sleep activity value generated using the sensed light and the sensed movement.

Example A28 includes the device of example A25, in which the device further includes a user interface including at least one of touch screen display or user buttons to receive user input, in which the set of quantitative metrics further includes a device usage value generated by the data processing unit based on tracking usage of the user interface associated with the user.

Example A29 includes the device of example A25, in which the data processing unit is further configured to compare the determined metric indicative of the current clinical state of the user to a binary threshold defining a range of stability and instability, and to produce a binary output indicative of the current clinical state of the user as being stable or unstable.

Example A30 includes the device of example A25, in which the data processing unit is further configured to determine a differential value between the determined metric indicative of the current clinical state of the user and a threshold; and to determine a level of change of social rhythmicy of the user's current clinical state based on the differential value.

Example A31 includes the device of example A25, in which the data processing unit is further configured to rank the quantitative metrics to assess importance of each metric in predicting stability of the determined metric indicative of the current clinical state from the data detected by the plurality of sensors.

Example A32 includes the device of example A25, in which the sensors are resident on a user device including a smartphone, a tablet, a smartwatch, or a smartglasses device, and the data processing unit is resident on one or more computers in communication with the user device via a communication network or link.

Example A33 includes the device of example A32, in which the one or more computers is configured to generate the set of quantitative metrics and to determine the metric indicative of the current clinical state.

Example A33 includes the device of example A32, in which the user device is configured to generate the set of quantitative metrics, and the one or more computers is configured to determine the metric indicative of the current clinical state.

In some embodiments in accordance with the present technology (example B1), a user device includes a memory; a processor; an ambient light sensor; and a microphone to receive ambient audio, in which the ambient light sensor senses ambient light and produces an ambient light signal, in which the microphone periodically samples ambient audio to produce sampled audio, and in which the processor uses the ambient light signal and the sampled audio to determine a present mental health condition of a user of the user device.

In some embodiments in accordance with the present technology (example B2), a method of monitoring a patient's current condition, implemented by a user device, includes sensing a physical parameter related to the patient's ambience; tracking usage data of the patient's use of the user device; and estimating, at least partly without explicit input from the patient, the patient's current condition from the physical parameter and the usage data.

Example B3 includes the method of example B2, in which the physical parameter includes one of the patient's location, sounds heard by the patient and ambient light near the patient.

Example B4 includes the method of example B2, in which usage data includes the patient's call log and/or text messaging usage.

Example B5 includes the method of example B2, in which the sensing the physical parameter includes sensing the patient's movement at various times of day.

Example B6 includes the method of example B5, further including, based on the patient's movement, estimating a state of the patient's circadian rhythm.

Implementations of the subject matter and the functional operations described in this patent document can be implemented in various systems, digital electronic circuitry, or in computer software, firmware, or hardware, including the structures disclosed in this specification and their structural equivalents, or in combinations of one or more of them. Implementations of the subject matter described in this specification can be implemented as one or more computer program products, i.e., one or more modules of computer program instructions encoded on a tangible and non-transitory computer readable medium for execution by, or to control the operation of, data processing apparatus. The computer readable medium can be a machine-readable storage device, a machine-readable storage substrate, a memory device, a composition of matter effecting a machine-readable propagated signal, or a combination of one or more of them. The term “data processing unit” or “data processing apparatus” encompasses all apparatus, devices, and machines for processing data, including by way of example a programmable processor, a computer, or multiple processors or computers. The apparatus can include, in addition to hardware, code that creates an execution environment for the computer program in question, e.g., code that constitutes processor firmware, a protocol stack, a database management system, an operating system, or a combination of one or more of them.

A computer program (also known as a program, software, software application, script, or code) can be written in any form of programming language, including compiled or interpreted languages, and it can be deployed in any form, including as a stand-alone program or as a module, component, subroutine, or other unit suitable for use in a computing environment. A computer program does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

The processes and logic flows described in this specification can be performed by one or more programmable processors executing one or more computer programs to perform functions by operating on input data and generating output. The processes and logic flows can also be performed by, and apparatus can also be implemented as, special purpose logic circuitry, e.g., an FPGA (field programmable gate array) or an ASIC (application specific integrated circuit).

Processors suitable for the execution of a computer program include, by way of example, both general and special purpose microprocessors, and any one or more processors of any kind of digital computer. Generally, a processor will receive instructions and data from a read only memory or a random access memory or both. The essential elements of a computer are a processor for performing instructions and one or more memory devices for storing instructions and data. Generally, a computer will also include, or be operatively coupled to receive data from or transfer data to, or both, one or more mass storage devices for storing data, e.g., magnetic, magneto optical disks, or optical disks. However, a computer need not have such devices. Computer readable media suitable for storing computer program instructions and data include all forms of nonvolatile memory, media and memory devices, including by way of example semiconductor memory devices, e.g., EPROM, EEPROM, and flash memory devices. The processor and the memory can be supplemented by, or incorporated in, special purpose logic circuitry.

It is intended that the specification, together with the drawings, be considered exemplary only, where exemplary means an example. As used herein, the singular forms “a”, “an” and “the” are intended to include the plural forms as well, unless the context clearly indicates otherwise. Additionally, the use of “or” is intended to include “and/or”, unless the context clearly indicates otherwise.

While this patent document contains many specifics, these should not be construed as limitations on the scope of any invention or of what may be claimed, but rather as descriptions of features that may be specific to particular embodiments of particular inventions. Certain features that are described in this patent document in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. Moreover, the separation of various system components in the embodiments described in this patent document should not be understood as requiring such separation in all embodiments.

Only a few implementations and examples are described and other implementations, enhancements and variations can be made based on what is described and illustrated in this patent document.

What is claimed is:

1. A method for passively monitoring a health condition of a subject using a mobile device, the method comprising:
producing a set of quantitative metrics based on one or more parameters including location, movement, and sound obtained from a mobile communications device associated with the subject, wherein the produced set of quantitative metrics includes a location cluster value, a travel distance value, a frequency of conversation value, and an activity value, and each of the quantitative metrics is over a respective predetermined time period; and
processing the set of quantitative metrics to determine a metric indicative of a current clinical state of the subject in connection with one or more measures including daily routines, mood or energy of the subject.

2. The method of claim 1, wherein the metric indicative of the current clinical state is determined at least partly without active input from the subject.

3. The method of claim 1, further comprising:
comparing the determined metric indicative of the current clinical state of the subject to a binary threshold defining a range of stability and instability; and
producing a binary output indicative of the current clinical state of the subject being stable or unstable.

4. The method of claim 1, further comprising:
determining a differential value between the determined metric indicative of the current clinical state of the subject and a threshold; and
evaluating the differential value to determine a level of change of social rhythmicy of the subject's current clinical state.

5. The method of claim 1, further comprising:
ranking the quantitative metrics to assess importance of each quantitative metric in predicting stability of the determined metric from the parameters.

6. The method of claim 1, further comprising:
sensing the parameters including the location, the movement, and the sound using a location sensor, a motion sensor, and a sound sensor, respectively, of the mobile communications device.

7. The method of claim 1, wherein the frequency of conversation is produced using the sensed sound, comprising:
sampling audio from a sound sensor of the mobile communications device;
determining human speech from other sound present in the sampled audio; and
discerning live human speech from non-live voices present in the sampled audio.

8. The method of claim 7, wherein the sensing the sound further comprises controlling the sound sensor to selectively sample the audio on a periodic or intermittent basis.

9. The method of claim 7, wherein the sampling the audio does not include recording speech, wherein the determining live human speech includes analyzing the sampled audio in real time including extracting one or more audio features including spectral content, regularity, volume, or pitch magnitudes and changes.

10. The method of claim 1, wherein the one or more parameters further includes light, and wherein the light is sensed using one or more of an ambient light sensor or camera of the mobile communications device.

11. The method of claim 10, further comprising:
producing a sleep activity value included in the set of quantitative metrics, the sleep activity value associated with the sensed light and the sensed movement.

12. The method of claim 1, further comprising:
tracking usage data of the subject's use of the mobile communication device; and
producing a quantitative metric associated with tracked usage data to include with the produced set of quantitative metrics, such that the produced set of quantitative metrics further includes a device usage value.

13. The method of claim 1, wherein the mobile communication device includes a smartphone, a tablet, a smartwatch, or a smartglasses device.

14. A device for passively monitoring a health condition, comprising:
a plurality of sensors including a location sensor, a motion sensor and a sound sensor, wherein the sensors detect location data, movement data, and sound data associated with a user of the device; and
a data processing unit including a memory to store data from the sensors and a processor configured to processes the location data, the movement data and the sound data to generate a set of quantitative metrics including a location cluster value, a travel distance value, a frequency of conversation value, and an activity value, in which each of the quantitative metrics is over a respective predetermined time period, and to determine a metric indicative of a current clinical state of the user in connection with one or more measures including daily routines, mood or energy of the user,
wherein the metric indicative of the current clinical state is determined at least partly without active input from the user.

15. The device of claim 14, wherein the device includes a smartphone, a tablet, a smartwatch, or a smartglasses device including a software application comprising program code executable by the processor and stored in the memory to provide instructions for the processor to generate the set of quantitative metrics and determine the metric indicative of the current clinical state.

16. The device of claim 14, wherein the plurality of sensors further includes one or more of an ambient light sensor or a camera to detect light data, and wherein the set of quantitative metrics further includes a sleep activity value generated using the sensed light and the sensed movement.

17. The device of claim 14, the device further including a user interface including at least one of touch screen display or user buttons to receive user input, wherein the set of quantitative metrics further includes a device usage value generated by the data processing unit based on tracking usage of the user interface associated with the user.

18. The device of claim 14, wherein the data processing unit is further configured to compare the determined metric indicative of the current clinical state of the user to a binary threshold defining a range of stability and instability, and to produce a binary output indicative of the current clinical state of the user as being stable or unstable.

19. The device of claim 14, wherein the data processing unit is further configured to determine a differential value between the determined metric indicative of the current clinical state of the user and a threshold; and to determine a level of change of social rhythmicy of the user's current clinical state based on the differential value.

20. The device of claim 14, wherein the data processing unit is further configured to rank the quantitative metrics to assess importance of each metric in predicting stability of the determined metric indicative of the current clinical state from the data detected by the plurality of sensors.
